(19) United States
(12) Patent Application Publication
(10) Pub. No.: US 2008/0103349 A1
(43) Pub. Date: May 1, 2008

(54) Therapeutic benefits of magnetic essential oil use

(75) Inventor: Victoria Marie Richards, Canton, GA (US)

Correspondence Address: Victoria Richards, 3640 Hickory Flat Highway, Canton, GA, 30115, US

(73) Assignee: Victoria Richards, Canton, GA (US)

(21) Appl. No.: 11/588,155

(22) Filed: Oct. 27, 2006

(51) Int. Cl. A61N 2/00; A61H 1/00

(52) U.S. Cl. 600/12; 601/17

(57) ABSTRACT

The use of essential oils and biocompatible magnets is described. The combination of these two treatments can result in weight loss experiences, reduced stress and other wellness feelings and experiences. Placing small powerful magnets on various places on the body and essential oils onto the body effects that improve wellness are seen.

### SUMMARY OF THE INVENTION

The present invention uses a combination of magnetic and essential oils to elicit beneficial effects. Placement of small powerful magnets on various body parts in combination with the use of certain essential oils can result in weight loss, reduced feelings of stress and other wellness characteristics. The placement of the magnets onto the body is accomplished using biocompatible adhesives. The specific placement of the magnets on the body is of importance to produce different effects. The use of essential oils in conjunction with the use of the magnets results in effects that are not seen or have not been described when either magnets or essential oils alone are used.

### BACKGROUND

The use of magnets and essential oils have been used alone to cause responses in the body that result in beneficial results for the individual. Until now the benefits of the combined use of magnets and essential oils have not be described.

### BRIEF DESCRIPTION OF THE FIGURES

FIG. 1. Identification the locations of the placement of magnets for the treatment of diarrhea.

FIG. 2. Identification of the locations for the placement of essential oils blends for the treatment of diarrhea.

FIG. 3. Identification the locations of the placement of magnets for the treatment of headaches.

FIG. 4. Identification of the anterior locations for the placement of essential oils blends for the treatment of headaches.

FIG. 5. Identification the posterior locations of the placement of essential oils for the treatment of headaches.

FIG. 6. Identification the locations of the placement of magnets for the treatment of stress.

FIG. 7. Identification of the locations for the placement of essential oils blends for the treatment of stress.

### DETAILED DESCRIPTION OF THE INVENTION

The present invention is a method to produce a therapeutic effect using a combination of magnetic fields and essential oils on the body. This combination can produce therapeutic effects including weight loss, increased libido, increased energy levels, and decreased susceptibility to illness and other benefits that is not seen with individual treatments. It is the combination of these two treatments simultaneously that produces a surprisingly effective treatment regime.

### Magnetic Field:

The magnetic field that is used in the present invention can be formed in any way that produces a detectable and effective field. This magnetic field may be produced using electromagnetic, permanent or temporary magnets. The magnetic field that is used in the present invention can be produced in a variety of ways depending on the effect that is desired.

In one embodiment electromagnets may be used to produce the magnetic field. Batteries or other small energy-producing device may power the electromagnets. The electromagnets may be miniaturized as described in U.S. Pat. No. 5,919,306 said description incorporated herein. A disadvantage of electromagnets is their expense and requirement for and energy source to maintain their magnetic property.

Temporary magnets may be used to produce the magnetic field. Temporary magnets are typically magnets made by exposing a ferromagnetic material to a strong external magnetic field for a period of time. This hysteresis effect allows the ferromagnetic material to retain a magnetic property for a period of time. The preferred manner to produce this hysteresis is by inserting the ferromagnetic material into the core of an electromagnet. Use of a strong permanent magnet may also be used to produce he hysteresis effect. A significant disadvantage of these types of magnets is that they are not powerful and lose their magnetic properties over time. This inactivation can result in a loss or a reduction of the therapeutic effects of the magnet and essential oil combination.

In a preferred embodiment the magnetic field is produced using one or more permanent magnets. Permanent magnets may be of any type that provides a substantial magnetic field that can be used in the present invention. Suitable types of permanent magnets include plastic magnets, alnico magnets, ceramic magnets, and rare earth magnets. Plastic magnets are comprised of a relatively new magnetic material and are non-metallic. Plastic magnets are typically comprised of a combination of polyaniline and tetracyanoquinodimethane forming a magnetic polymer. One advantage of the use of these types of magnets in the present invention is their inherent biocompatibility.

Metallic magnets include alnico, ceramic and rare earth magnets. Alnico magnets are comprised of various mixtures of materials including iron, cobalt, nickel, aluminum and copper. These magnets are typically stronger than ceramic or ferrite magnets but not as strong as rare earth magnets. A disadvantage of alnico magnets is their propensity to demagnetize by proximity to other magnets, by being subjected to impact collisions and by not keeping the magnet with its keeper. In a preferred embodiment of the present invention either ceramic or rare earth magnets are used. Ceramic or ferrite magnets are comprised generally of strontium carbonate and iron oxide. These magnets are particularly suitable for use in the present invention due to their low cost, good magnetism, good corrosion resistance, and ease of coating. Rare earth magnets include neodymium and samarium-cobalt magnets. A neodymium magnet is comprised of a combination of neodymium, iron and boron. A samarium-cobalt magnet is comprised of samarium, cobalt and iron. Rare earth magnets are very strong magnets but exhibit poor corrosion characteristics that encourage coating of the magnets.

Alnico, ceramic and rare earth magnets may be coated or plated with materials that are different from the magnetic material. Certain coating materials may improve the biocompatibility of the magnets by coating the magnet surface with a non-reactive material that minimizes irritation and reactions with the skin. Suitable coatings include titanium, stainless steel, nickel, zinc, silver and gold.

Magnets used in the present invention may be of any shape or size. Preferred magnets range between 0.1-0.4 inches in diameter. While any shaped magnet may be used, preferred shapes are round and oval with no sharp corners. The gauss values for magnets in the present invention typically range between about 400-9,000 gauss.

The magnets of the present invention can be adhered to the skin with a non-permanent adhesive. Preferred adhesives are hypoallergenic and exhibit low irritation characteristics. Biocompatible adhesives have been describe in U.S. Pat. Nos. 4,527,087, 4,539,996, 4,554,924, 4,848,353, and 5,012,810 and are incorporated herein by reference. The adhesive may be directly applied to the magnet or applied to a plaster unto which the magnet is attached. The plaster is then affixed with the magnet to the body. The plaster is useful in providing an easily transferred article because of its increased size.

The magnets are placed on the body according to traditional acupuncture sites as described in “Chinese Herbal Medicine: Materia Medica, Third Edition by Dan Bensky”, “The Treatment of Modern Western Diseases with Chinese Medicine: A Textbook and Clinical Manual by Bob Flaws”, “Practical Diagnosis in Traditional Chinese Medicine by Tieh-Tao Teng”, “A Manual of Acupuncture by Peter Deadman”, and “Diagnosis in Chinese Medicine: A Comprehensive Guide by Giovanni Maciocia” and incorporated herein by reference. These sites relate to points that can be used to attach magnets for specific treatments.

### Essential Oils:

The essential oils used in the present invention are oils from any plant source that is available. Expression, cold pressing, solvent extraction, steam distillation or any other procedure that results in an essential oil may produce the essential oils. Preferred essential oils are therapeutic essential oils. Therapeutic essential oils are undiluted by adulterating liquids. These adulterating liquids include synthetic fragrance raw materials, solvents, vegetable oil diluents, petroleum oil diluents and other materials that have not been extracted from a plant used for the production of essential oils. In a preferred embodiment the therapeutic essential oils conform to the essential oils described in Huiles essentielles available from ANFOR, the European standards organization.

The following examples serve to show how the combination of the use of magnets and essential oils on the body may be arranged to provide therapeutic effects. While there are many other potential conformations and combinations of magnets and essential oil blends that can cause therapeutic effects the examples below show the basic procedure that describes the present invention.

### EXAMPLE 1

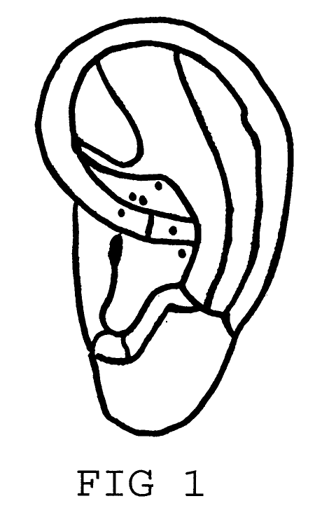

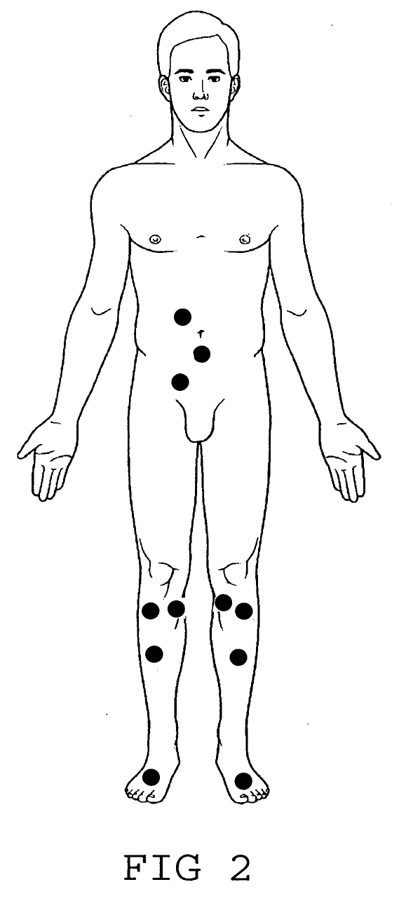

This procedure reduces the frequency and severity of food related diarrhea. A blend of 5 essential oils was made by mixing 14 parts basil, 10 parts chamomile, 40 parts peppermint, 9 parts thyme, 10 parts eucalyptus and 17 parts ginger until homogeneous. Magnets were placed on the ear at the locations shown in FIG. 1. The blend of essential oils was rubbed on the body at the locations identified in FIG. 2, the bottoms of the feet and behind the ear opposite to the magnets.

### EXAMPLE 2

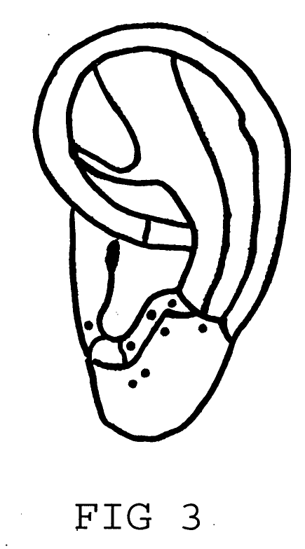

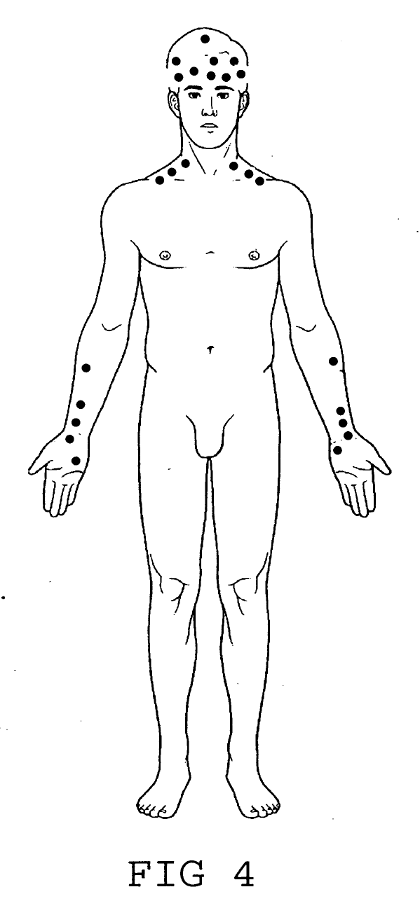

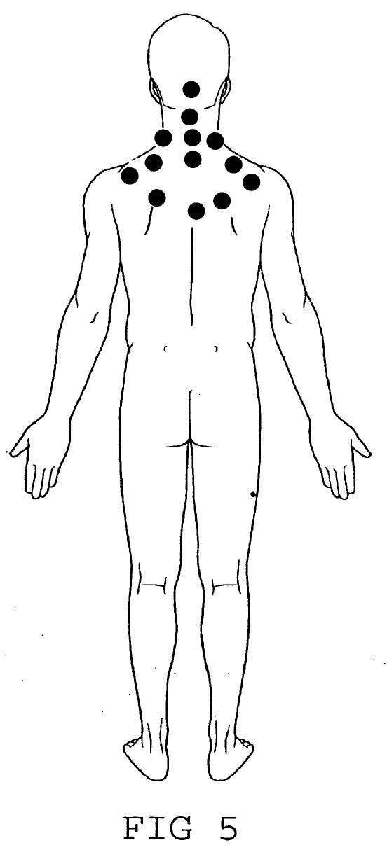

This procedure reduces the frequency and severity of headaches. A blend of 4 essential oils was made by mixing 30 parts lavender, 10 parts wintergreen, 40 parts peppermint and 20 parts basil until homogeneous. Magnets were placed on the ear at the locations shown in FIG. 3. The blend of essential oils was rubbed on the body at the locations identified in FIGS. 4 and 5, the bottoms of the feet and behind the ear opposite to the magnets.

### EXAMPLE 3

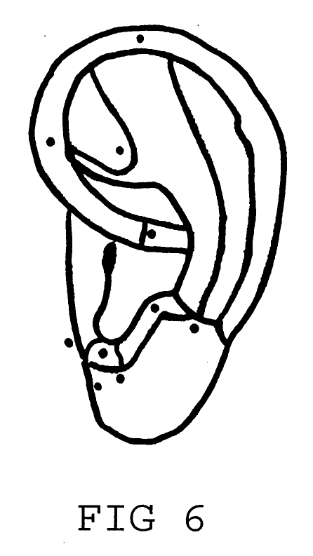

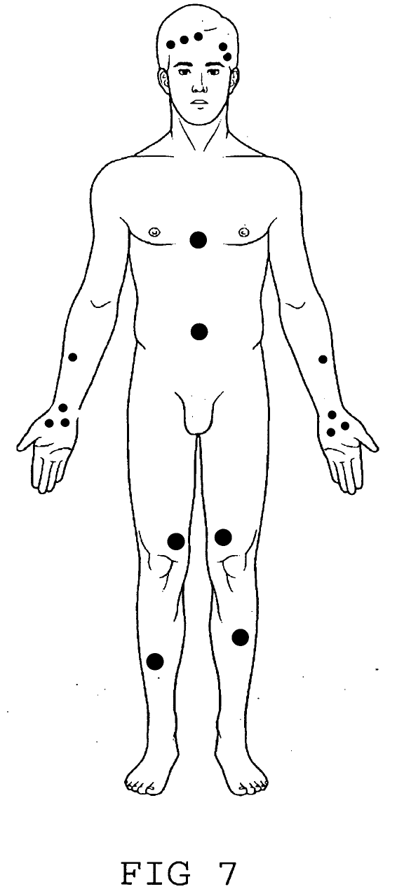

This procedure reduces anxiety and stress. A blend of 5 essential oils was made by mixing 25 parts lavender, 12 parts chamomile, 25 parts geranium, 15 drops sandalwood and 25 parts cedarwood until homogeneous. Magnets were placed on the ear at the locations shown in FIG. 6. The blend of essential oils was rubbed on the body at the locations identified in FIG. 7, the bottoms of the feet and behind the ear opposite to the magnets.

Typically, the magnets are placed on the ear in locations that are identified as important sites in Acupuncture, Acupressure and Reflexology. In a preferred embodiment multiple magnets are used to elicit the magnetic field. Because of the variation in individuals, the individual magnet strengths may vary between 400-9,000 gauss. The magnets are changed at the end of each week of treatment when the fresh addition of essential oil is administered. The essential oil may be administered on a periodic timetable if the effect requires it.

These examples describe several applications for the use of the present invention. Other applications using similar combinations of magnets and essential oils on the body, can affect other therapeutic effects. These effects may include the common cold, bronchitis, hypertension, obesity, myopia, herpes zoster, asthma and tonsillitis.

I claim:

1. A method to produce a therapeutic effect using a combination of magnetic fields and essential oils on the body.

2. The method of claim 1 wherein the magnetic field is produced using permanent magnets.

3. The method of claim 2 wherein the magnet is a ceramic or rare earth magnet.

4. The method of claim 3 wherein the permanent magnet is coated.

5. The method of claim 4 wherein the coating is titanium, stainless steel, gold or silver.

6. The method of claim 2 wherein the magnets are placed primarily on the ear.

7. The method of claim 1 wherein the essential oils are therapeutic essential oils.

8. The method of claim 2 wherein the magnets are placed on the ears.

8. The method of claim 1 wherein the therapeutic effect includes a physiological effect.

9. The method of claim 1 wherein essential oil is placed on the feet.

10. The method of claim 1 wherein the treatment includes an electromagnetic stimulation.
